(19) United States
(12) Patent Application Publication
(10) Pub. No.: US 2026/0158247 A1
(43) Pub. Date: Jun. 11, 2026

(54) BALLOON CATHETER

(71) Applicant: MYODYNAMICS, LLC, Englewood Cliffs, NJ (US)

(72) Inventors: Ashwani Sastry, Orlando, FL (US); Sreejit Nair, Pembroke Pines, FL (US)

(21) Appl. No.: 18/973,949

(22) Filed: Dec. 9, 2024

(51) Int. Cl. A61M 25/10; A61F 2/958

(52) U.S. Cl. CPC A61M 25/104; A61F 2/958; A61M 25/1002

(57) ABSTRACT

The present invention relates to a balloon catheter. The balloon catheter includes a tubular member and an expandable member. The tubular member defines an inflation lumen therein at least to facilitate a fluid flow. The expandable member is mounted to a distal end section of the tubular member and defines a chamber therein. The chamber fluidically communicates with the inflation lumen and enables the expandable member to transition between an inflated configuration and a deflated configuration. The expandable member is configured with a proximal diametrical size and a distal diametrical size. The distal diametrical size is larger than the proximal diametrical size. The expandable member increases from the proximal diametrical size to the distal diametrical size, along a longitudinal axis thereof, and is adapted to dilate the body lumen in the inflated configuration.

### TECHNICAL FIELD

The present disclosure relates to medical devices, and more particularly, to a balloon catheter including an expandable member designed to adapt to a natural profile of a vessel within a body lumen during retrograde access.

### BACKGROUND

Endovascular surgery utilizing balloon catheters has revolutionized the treatment of vascular conditions, offering minimally invasive alternatives to traditional surgical procedures. During these interventions, a balloon catheter is inserted into the vascular system through a small access point, typically in the groin or wrist. Once positioned at the site of a blockage or narrowing, the catheter's balloon is inflated to dilate the affected blood vessel, effectively restoring proper blood flow. This technique not only minimizes trauma to surrounding tissues but also reduces recovery time and hospital stays compared to open surgery. Additionally, balloon angioplasty can be combined with stent placement to provide long-term support for the treated vessel.

Access to balloon catheter procedures can be achieved through either antegrade or retrograde approaches, each tailored to the specific clinical needs of the patient. Antegrade access involves inserting the catheter from a proximal site, such as the radial or femoral artery, allowing for straightforward navigation in the direction of blood flow. This method is commonly used for direct interventions in coronary arteries and other vascular regions. Conversely, retrograde access entails introducing the catheter from a distal site, typically against the natural flow of blood, such as from the femoral artery toward the heart or other internal organs. This approach is particularly advantageous in cases where direct access is challenging, enabling effective treatment of obstructions or lesions.

The balloon of the catheter is designed with both non-tapered and tapered profiles. Conventional non-tapered balloons often lack the necessary customization, leading to potential over-dilation or under-dilation of vessel segments. This limitation makes them less suitable for treating tapering segments during angioplasty, especially when approached retrogradely. On the other hand, tapered balloons can provide a better fit; however, when inserted retrogradely, the distal tapered end aligns with the wider section of the blood vessel, while the less tapered proximal end opposes the narrower segment located further downstream. This mismatch can hinder the effective dilation of tapering vasculature, potentially compromising the success of angioplasty procedures.

Therefore, there is a need to design a balloon catheter that overcomes one or more limitations stated above, in addition to providing other technical advantages.

### SUMMARY

Various embodiments of the present disclosure provide a balloon catheter including an expandable member adapted to conform to a tapered profile of a vessel of a body lumen when introduced through retrograde access.

In an embodiment, a balloon catheter is disclosed. The balloon catheter includes a tubular member and an expandable member. The tubular member defines an inflation lumen therein at least to facilitate a fluid flow. The tubular member includes a proximal end section and a distal end section. The proximal end section is adapted to be fluidically coupled to a fluid source, and the distal end section is adapted to be introduced into a body lumen at least through retrograde access thereof. The expandable member is mounted to the distal end section of the tubular member and defines a chamber therein. The chamber fluidically communicates with the inflation lumen and enables the expandable member to transition between an inflated configuration and a deflated configuration. The expandable member is configured with a proximal diametrical size and a distal diametrical size. The distal diametrical size is larger than the proximal diametrical size. The expandable member increases from the proximal diametrical size to the distal diametrical size along a longitudinal axis thereof and is adapted to dilate the body lumen in the inflated configuration.

In another embodiment, a method for dilating a body lumen is disclosed. The method includes providing a balloon catheter. The balloon catheter includes a tubular member and an expandable member. The expandable member is adapted to be mounted to the tubular member. The expandable member is configured with a proximal diametrical size and a distal diametrical size. The distal diametrical size is larger than the proximal diametrical size. The expandable member increases from the proximal diametrical size to the distal diametrical size along a longitudinal axis thereof. The method further includes advancing the balloon catheter into the body lumen through retrograde access thereof. Furthermore, the method includes inflating the expandable member to conform to a profile of the body lumen.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or tool and instrumentalities disclosed herein. Moreover, those in the art will understand that the drawings are not to scale. Wherever possible, like elements have been indicated by identical numbers:

FIG. 1A illustrates a schematic representation of a subject undergoing endovascular procedures, in accordance with an example embodiment of the present disclosure;

FIG. 1B illustrates a schematic representation of an exemplary balloon catheter introduced into a foot of the subject, in accordance with an example embodiment of the present disclosure;

FIG. 2A illustrates a schematic representation of the balloon catheter, in accordance with at least some embodiments of the present disclosure;

FIG. 2B illustrates a sectional view of the balloon catheter depicted in FIG. 2A;

FIG. 2C illustrates a sectional view of section A-A′ of a catheter body of the balloon catheter depicted in FIG. 2A;

FIG. 3A illustrates a perspective view of an expandable member of the balloon catheter depicted in FIGS. 1A-1B and 2A-2B, in accordance with at least some embodiments of the present disclosure;

FIG. 3B illustrates a perspective sectional view of the expandable member depicted in FIG. 3A;

FIG. 4A illustrates a sectional view of an expandable member depicted in FIGS. 3A-3B, in accordance with one embodiment of the present disclosure;

FIG. 4B illustrates a sectional view of an expandable member depicted in FIGS. 3A-3B, in accordance with another embodiment of the present disclosure;

FIG. 5 illustrates a sectional view of an expandable member depicted in FIGS. 3A-3B, in accordance with another embodiment of the present disclosure;

FIG. 6A illustrates a sectional view of an expandable member depicted in FIGS. 3A-3B, in accordance with another embodiment of the present disclosure;

FIG. 6B illustrates a sectional view of an expandable member depicted in FIGS. 3A-3B, in accordance with yet another embodiment of the present disclosure; and

FIG. 7 illustrates a flow diagram of a method for dilating the body lumen, in accordance with various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

The term “distal” refers to a portion farthest away from a user when introducing a device into a patient.

The term “proximal” refers to a portion closest to the user when introducing the device to the patient.

Various examples of the present disclosure provide a balloon catheter. The balloon catheter mainly includes a catheter body, an expandable member mounted to the catheter body, and a guidewire adapted to extend at least partially through the catheter body and fully through the expandable member. An outer tubular member of the catheter body facilitates the delivery of fluid within a chamber of the expandable member, and an inner tubular member of the catheter body serves as a conduit for the guidewire. The expandable member is configured in a manner that conforms to the natural taper of the arteries or veins when inserted from the retrograde access. The expandable member is configured with a proximal diametrical size and a distal diametrical size. The distal diametrical size is larger than the proximal diametrical size, and therefore the expandable member defines a geometry that increases from the proximal diametrical size to the distal diametrical size, along a longitudinal axis thereof. By conforming to the shape of the vessel, such as the blood vessel, the expandable member allows for targeted treatment while minimizing damage to surrounding tissues, making it a vital tool in cardiovascular and other interventional procedures. The compliance characteristics of the expandable member are essential for its performance in vascular procedures of retrograde access, referring to its ability to deform and adapt in response to applied pressure. Therefore, in one embodiment, a wall thickness of the expandable member is maintained constant along its longitudinal axis, and in another embodiment, a wall thickness is reduced from its proximal end to distal end. Further, in another embodiment, to vary the compliance characteristics of the inflation of the expandable member, the material composition of the expandable member is varied from the proximal end to the distal end. In yet another embodiment, to vary the compliance characteristics of the inflation, the expandable member is made of two or more layers, each having a different material.

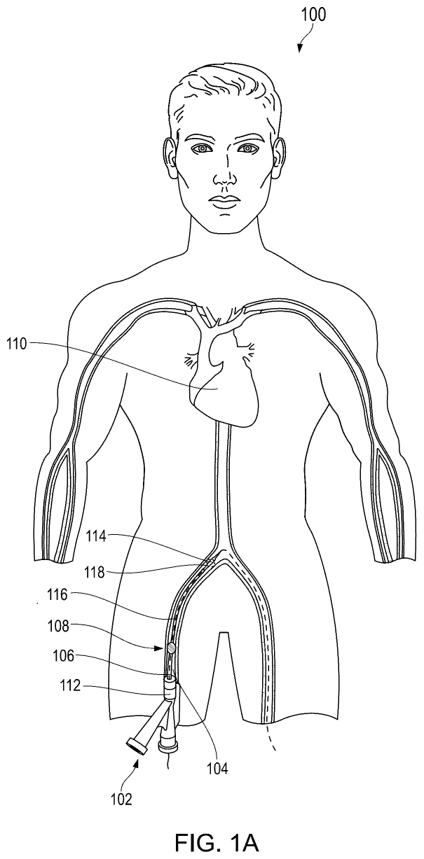

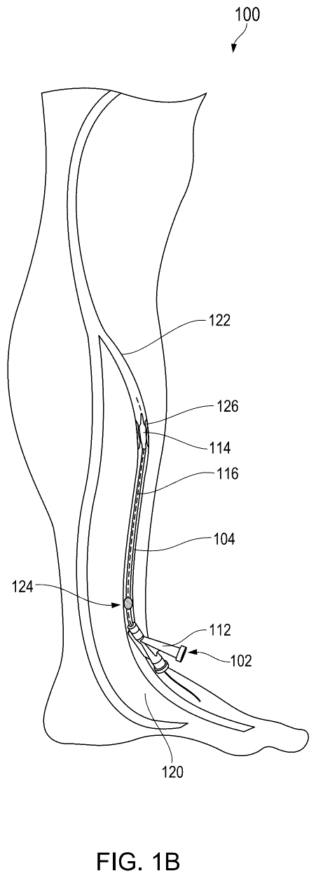

Various embodiments of the present disclosure are described with reference to FIGS. 1A-1B to FIG. 7.

FIG. 1A illustrates a schematic representation of a subject 100 (alternatively referred to as ‘patient 100’) undergoing endovascular procedures, in accordance with an example embodiment of the present disclosure. In the representative example, an exemplary balloon catheter 102 (also referred to as ‘catheter 102’) is introduced into a body lumen 104 (also referred to as ‘lumen 104’) of a vessel (e.g., a blood vessel 106), through an access point, such as a vascular access point 108 of the subject 100, using a retrograde approach. The retrograde approach refers to the approach of entering the catheter 102 into the lumen 104 of a vessel, such as the blood vessel 106, from the vascular access point 108 located at a distal site thereof. This allows the catheter 102 to travel upstream against the normal direction of blood flow, which is crucial for accessing the internal organs of the subject 100 during various procedures, such as cardiac catheterization. By navigating the balloon catheter 102 through the vascular system, clinicians can target specific areas, such as, but not limited to, the coronary arteries, to address conditions like arterial blockages or stenosis. Those skilled in the art would appreciate that the size and location of the vascular access point 108 may vary depending on the specific procedure. For instance, when performing the retrograde approach to treat the coronary vasculature of a heart 110, the balloon catheter 102 is advanced from the femoral artery into the descending aorta, then into the coronary arteries.

The balloon catheter 102 primarily includes a catheter body 112, an expandable member 114 mounted to the catheter body 112, and a guidewire 116 adapted to extend at least partially through the catheter body 112 and fully through the expandable member 114. The catheter body 112 provides structural support and rigidity, allowing it to navigate through the blood vessel 106. Without limiting the scope of the disclosure, the catheter body 112 facilitates the delivery of fluid, medications, or contrast agents to a targeted area 118, serves as a conduit for the guidewire 116 and the expandable member 114, and enhances maneuverability within the blood vessel 106. The expandable member 114, mounted to the catheter body 112, is an inflatable member designed to expand within the blood vessel 106 or another body lumen at a predefined configuration. Upon positioning the balloon catheter 102 at the targeted area 118 of the blood vessel 106, the expandable member 114 can be inflated to exert a radial force against the vessel walls (e.g., the wall of the blood vessel 106), effectively dilating the targeted area 118. The geometrical configuration of the expandable member 114 is such that, when inflated, it conforms to the tapered profile of the blood vessel 106 upon being introduced through retrograde access. Specifically, the expandable member 114 conforms to the natural taper of the arteries or veins (e.g., the blood vessel 106) when inserted from the retrograde access. By conforming to the shape of the vessel, such as the blood vessel 106, the expandable member 114 allows for targeted treatment while minimizing damage to surrounding tissues, making it a vital tool in cardiovascular and other interventional procedures.

Additionally, the expandable member 114, adapted to conform to the tapered profile (increasing diameter from a proximal region to a distal region) of the vessel (e.g., the blood vessel 106), may be used in interventions, such as angioplasty, where it is inflated to widen narrowed or blocked arteries, restoring blood flow using the retrograde access. Furthermore, the expandable member 114 can also be employed to compress plaque against vessel walls, deploy stents, or deliver medications directly to a specific site, without departing from the scope of the disclosure. This minimally invasive approach reduces recovery time and enhances patient outcomes compared to traditional surgical interventions, underscoring the significance of retrograde access in modern endovascular procedures.

In other examples, the catheter 102 is suitable for retrograde access to various arterial and vascular structures in the lower extremities, including the posterior tibial artery, dorsalis pedis artery, anterior tibial artery, and peroneal artery, as well as veins like the great saphenous vein and small saphenous vein. One such example is depicted below in FIG. 1B.

FIG. 1B illustrates a schematic representation of the exemplary balloon catheter 102 introduced into a foot 120 of the subject 100, in accordance with an example embodiment of the present disclosure. As shown, the catheter 102 is introduced into artery 122 of the foot 120 through retrograde access. The guidewire 116 of the catheter 102 is inserted into the artery 122 at an entry point 124 of the foot 120. The catheter body 112, along with the expandable member 114, is advanced into the artery 122 toward a constriction 126 to facilitate dilation of the artery 122.

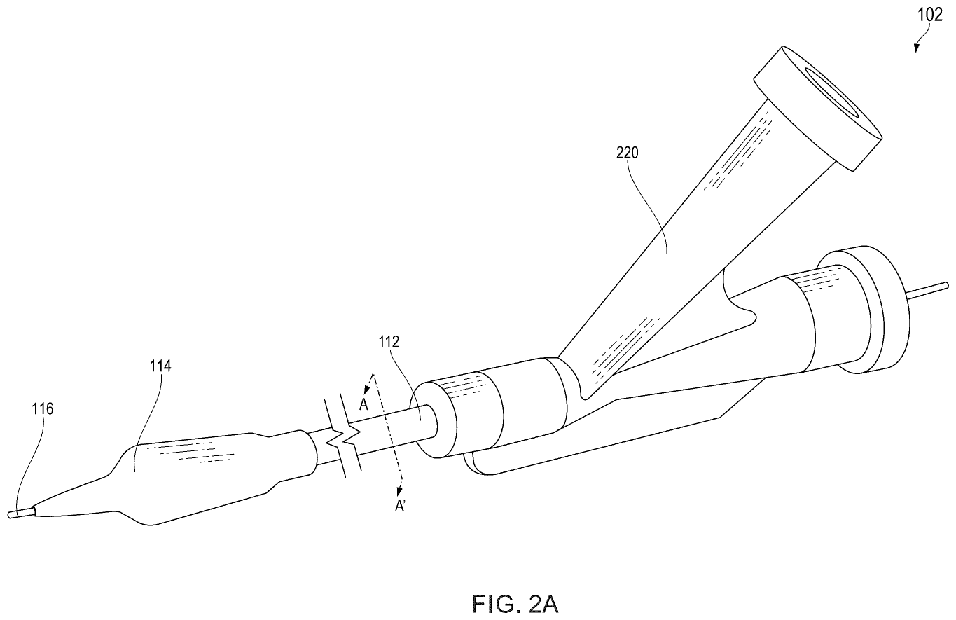

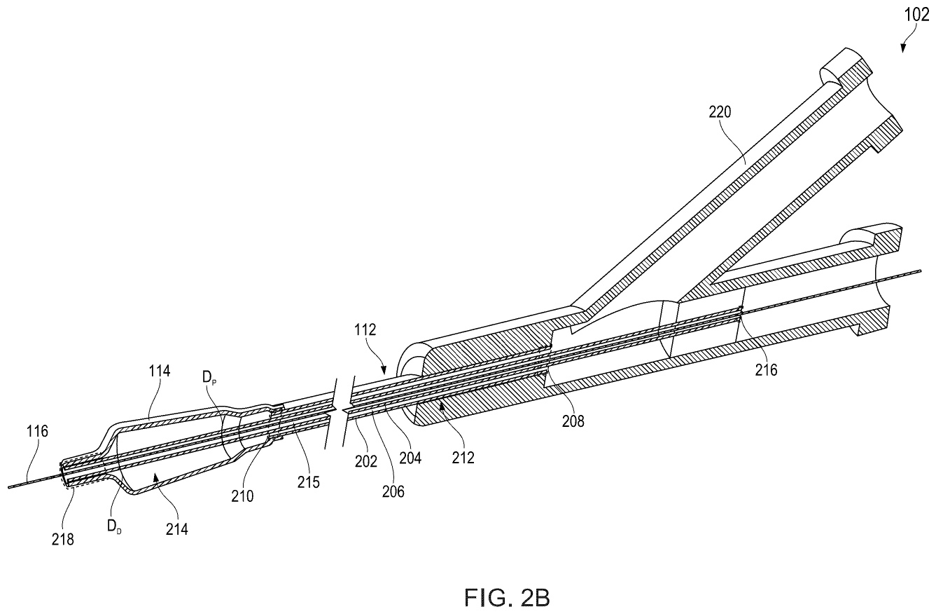

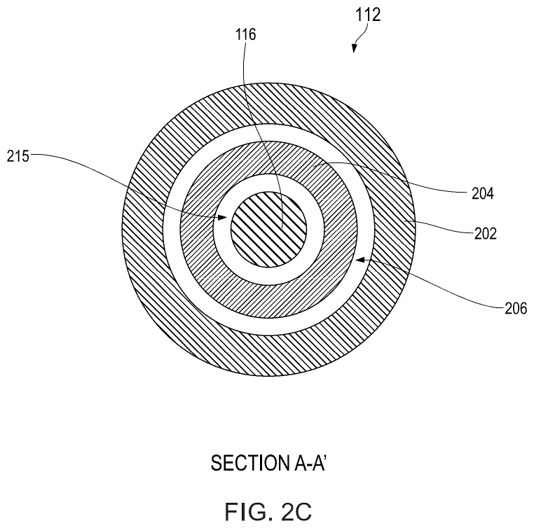

FIG. 2A illustrates a schematic representation of the balloon catheter 102, in accordance with at least some embodiments of the present disclosure. FIG. 2B illustrates a sectional view of the balloon catheter 102 depicted in FIG. 2A. FIG. 2C illustrates a sectional view of section A-A′ of the catheter body 112 depicted in FIG. 2A. The balloon catheter 102 includes, inter alia, the catheter body 112, the expandable member 114 adapted to be mounted to the catheter body 112, and the guidewire 116 adapted to extend at least partially through the catheter body 112 and fully through the expandable member 114.

The catheter body 112 is designed to advance over the guidewire 116 through the vascular pathway to reach the targeted area 118, while the expandable member 114 remains in an inflated or deflected configuration. The catheter body 112, designed to serve one or more purposes, may be constructed from one or more tubular members. In a specific embodiment, as shown in FIGS. 2B and 2C, the catheter body 112 is constructed from a tubular member 202 (also referred to as ‘outer tubular member 202’) and an inner tubular member 204. The outer tubular member 202 is configured as a hollow structural member defining a space therein, referred to as an inflation lumen 206.

As shown in FIG. 2C, the outer tubular member 202 encases the inner tubular member 204, creating a space between them that defines the inflation lumen 206. In other configurations, the location and position of the inflation lumen 206 may vary, depending on the configuration of the outer tubular member 202 and the inner tubular member 204. For instance, the outer tubular member 202 may be positioned alongside the inner tubular member 204, with the inflation lumen 206 then being defined by the lumen of the outer tubular member 202. The inflation lumen 206 facilitates the fluid flow there through.

The outer tubular member 202 has a proximal end section 208, a distal end section 210, and an intermediate section 212 between the proximal end section 208 and a distal end section 210. The proximal end section 208 is adapted to be fluidically coupled to a fluid source (not shown). On the other hand, the distal end section 210, along with the expandable member 114, is adapted to be introduced into the body lumen 104 of the patient 100 through retrograde access. The intermediate section 212 enables the fluid flow from the proximal end section 208 to the distal end section or vice versa.

The expandable member 114 is adapted to be mounted to the distal end section 210 of the outer tubular member 202. The expandable member 114 may be mounted to the distal end section 210 of the outer tubular member 202 using an appropriate method that ensures a leak proof connection. Without limiting the scope of the disclosure, the expandable member 114 may be adhesively or thermally bonded to the distal end section 210 of the outer tubular member 202.

The expandable member 114, having a balloon like construction, defines a chamber 214 therein. The chamber 214 is a flexible boundary that allows insertion and withdrawn of the fluid. The chamber 214 fluidically communicates with the inflation lumen 206 and enables the expandable member 114 to transition between the inflated configuration and the deflated configuration thereof. A person skilled in the art would appreciate that the fluid is being supplied and withdrawn from the chamber 214 through a pressure difference. For instance, the inflation lumen 206 can supply an inflation medium (i.e., fluid) within the chamber 214 under positive pressure. In contrast, the inflation lumen 206 may receive the inflation medium from the chamber 214 under negative pressure (i.e., vacuum pressure). The expandable member 114 can thus be inflated and deflated, depending on the fluid supplied or extracted from the chamber 214.

The expandable member 114, designed to conform to the natural taper (increasing diameter from a proximal region to a distal region) of the vessel when inserted from retrograde access, is configured with a proximal diametrical size D_{P} and a distal diametrical size D_{D}. In a specific embodiment, the distal diametrical size D_{D} is larger than the proximal diametrical size D_{P}. This implies that the expandable member 114 increases from the proximal diametrical size D_{P} to the distal diametrical size D_{D}, along a longitudinal axis L thereof, and is adapted to dilate the body lumen 104 of the subject 100 through retrograde access. Herein, the inflated configuration of the expandable member 114 is adapted to conform to a tapered profile of the vessel (e.g., blood vessel 106) of the body lumen 104 when introduced through the retrograde access. The geometrical configuration (i.e., increase in diameter from the proximal diametrical size D_{P} to the distal diametrical size D_{D}, along the longitudinal axis L) of the expandable member 114 of the present disclosure is reverse to the geometrical configuration of a conventional tapered balloon (not shown) which the diameter increases decreases from a proximal end to a distal end thereof. In other words, the expandable member 114 of the present disclosure configures a reverse tapered balloon. This allows the practitioner to treat the vessel of the subject 100 from retrograde access.

In a specific embodiment, the inner tubular member 204 is configured as a hollow structural member defining a ‘guidewire lumen 215’ therein. The inner tubular member 204 extends at least partially through the outer tubular member 202 and fully through the expandable member 114. In the depicted example, the inner tubular member 204 extends fully through each of the outer tubular member 202 and the expandable member 114. Alternatively, based on the specific design requirements, the inner tubular member 204 may extend partially through the outer tubular member 202 and/or the expandable member 114. The inner tubular member 204 has a proximal end region 216 and a distal end region 218. The proximal end region 216 is configured to receive the guidewire 116 and the distal end region 218 is configured to allow withdrawal of the guidewire 116. In other words, the guidewire 116 is adapted to be inserted from the proximal end region 216, passing through the guidewire lumen 215, and extracted from the distal end region 218.

The inner tubular member 204 of the can be designed in various configurations. For example, the inner tubular member 204 of the depicted design features an over-the-wire (OTW) configuration. This configuration allows for the independent advancement or retraction of the guidewire 116 and the catheter body 112 or enables the balloon catheter 102 to be navigated along a path established by the guidewire 116, ensuring precise placement during procedures. Alternatively, the inner tubular member 204 can feature a rapid exchange (RX) configuration. This configuration may allow for a quick and efficient exchange of the guidewire 116 without requiring the entire catheter 102 to be removed from the vascular area.

A person skilled in the art would appreciate that in each of the OTW and RX configurations, the inner tubular member 204 can be designed with either a single-lumen or multi-lumen arrangement. This versatility enables the balloon catheter 102 to accommodate various functionalities, such as the simultaneous delivery of multiple therapeutic agents or the integration of additional tools, enhancing its utility in complex procedures. A multi-lumen design can also facilitate improved flow dynamics and the ability to perform multiple tasks concurrently, optimizing procedural efficiency and patient outcomes. In an embodiment, the guidewire lumen 215 can be constructed with a thin membrane that offers sufficient strength to prevent the guidewire 116 from penetrating while minimizing any increase in the cross-sectional profile of the inner tubular member 204. Alternatively, the lumen may feature a multilayer construction that includes, but is not limited to, a layer of Nylon-L25, a bonding layer such as Prim, and a layer of high-density polyethylene (HDPE). Those skilled in the art will understand that various configurations and materials can be utilized without deviating from the scope of this disclosure, allowing for customization based on specific performance requirements and application needs.

It is to be noted that the inner tubular member 204 and the outer tubular member 202 can be arranged in various configurations. In the illustrated configuration, the inner tubular member 204 is positioned coaxially within the outer tubular member 202. Alternatively, in another configuration, the inner tubular member 204 can be arranged eccentrically relative to the outer tubular member 202. Additionally, in some embodiments, either the inner tubular member 204 or the outer tubular member 202 may be designed to allow for torque, facilitating the rotation of the catheter 102. Furthermore, without deviating from the scope of the disclosure, both members (i.e., the inner tubular member 204 and the outer tubular member 202) may also feature embedded reinforcing elements, such as coils or braided structures.

The outer tubular member 202 and the inner tubular member 204 may be manufactured from several materials. For example, the outer tubular member 202 and the inner tubular member 204 may be made of metals, metal alloys, polymers, metal-polymer composites, or any other suitable material, without limiting the scope of the disclosure. Further, it is contemplated that the stiffness of the outer tubular member 202 and/or the inner tubular member 204 may be modified to form the balloon catheter 102 for use in various vessel diameters and various locations with the vascular tree.

In the depicted example, the proximal tubular section 208 of the outer tubular member 202 and the proximal end region 216 of the inner tubular member 204 may be coupled to a hub 220 of the catheter 102. In the depicted example, the hub 220 has a Y-shaped profile, with one branch ending in a luer connector (not shown) to receive the fluid from the fluid source to fluidically interact with the proximal end section 208, and another branch equipped with a separate hemostatic valve (not shown) coupled to the proximal end region 216 for the guidewire 116. A conventional device, such as but not limited to an indeflator, syringe, etc., can be attached to the luer connector to introduce fluid into the inflation lumen 206. Additionally, a locking mechanism may be included to secure the operating position of the indeflator or syringe. The geometrical configuration and design feature of the hub 220 is well-known in the art, and therefore not extensively discussed here for the sake of brevity.

It may also be noted that the balloon catheter 102 is shown to have included the above-stated parts, however, those skilled in the art would appreciate that the balloon catheter 102 includes other parts (e.g., flexible tubular strain relief, hypotube, etc.) which may not be relevant for explaining the present disclosure and hence are not shown and described.

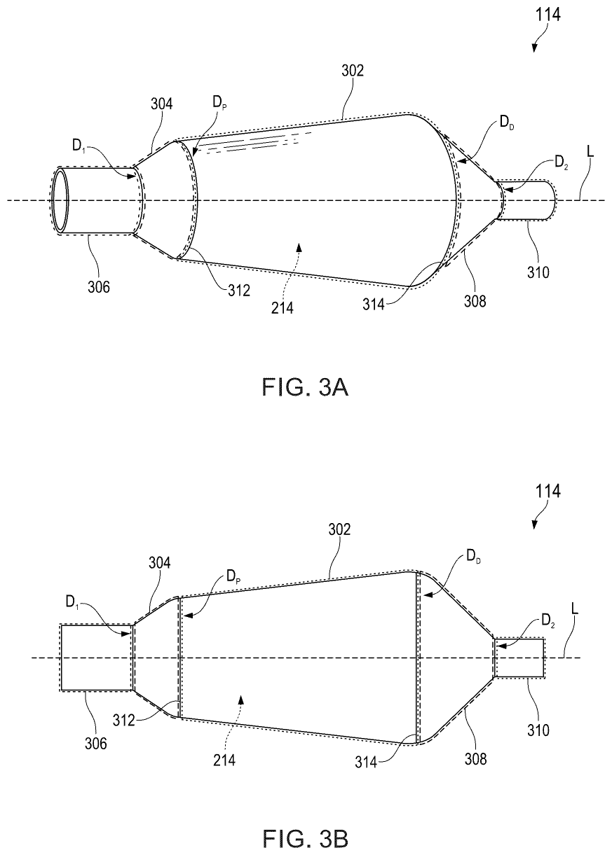

FIG. 3A illustrates a perspective view of the expandable member 114 depicted in FIGS. 1A-1B and 2A-2B, in accordance with at least some embodiments of the present disclosure. FIG. 3B illustrates a perspective sectional view of the expandable member 114 depicted in FIG. 3A. FIGS. 3A and 3B depict a detailed configuration of the expandable member 114. The expandable member 114 includes a body portion 302, a proximal conical portion 304, a proximal collar portion 306, a distal conical portion 308, and a distal collar portion 310. The body portion 302 is a part of the expandable member 114 that is designed to come in contact with the vessel wall, through retrograde access, to perform the desired function, such as but not limited to dilating a narrowed blood vessel, compressing plaque against the vessel walls, or delivering medications through retrograde access. The body portion 302 features a proximal end 312 and a distal end 314. The proximal end 312 is configured with the proximal diametrical size D_{P} and the distal end 314 is configured with the distal diametrical size D_{D}. In each of the embodiments of the disclosure, the distal diametrical size D_{D} is larger than the proximal diametrical size D_{P}. The body portion 302 increases from the proximal diametrical size D_{P} to the distal diametrical size D_{D}. However, depending on the design requirements, a gradient (i.e., slope) of increase of diameter from the proximal diametrical size D_{P} to the distal diametrical size D_{D} may vary in various embodiments of the disclosure.

The proximal cone portion 304 is primarily configured to separate the body portion 302 from the proximal collar portion 306, without limiting the scope of the disclosure. The proximal cone portion 306 converges proximally, along the longitudinal axis L, from the proximal diametrical size D_{P} to a first diametrical size D₁. The first diametrical size D₁ is smaller than the proximal diametrical size D_{P}. Convergence angle of the proximal cone portion 306 may depend on several factors, such as geometrical parameters (length and diameter) of the body portion 302, material properties (e.g., elasticity, stiffness, etc.) of the expandable member 114, the diameter of the distal end section 210, etc.

The proximal collar portion 306 extends proximally, along the longitudinal axis L, from the first diametrical size D₁. The proximal collar portion 306 is adapted to be mounted to the distal end section 210 of the outer tubular member 202. Depending on the geometrical profile of the distal end section 210 of the outer tubular member 202, the proximal collar portion 306 may be configured with a substantially uniform diameter or variable diameter, along the longitudinal axis L. The proximal collar portion 306 may be mounted to the distal end section 210 through a suitable leakproof technique. In one example, the proximal collar portion 308 may be adhesively bonded to the distal end section 210. In another example, the proximal collar portion 306 may be thermally bonded to the distal end section 210. In each of these examples, the specific bonding location of the distal end section 210 can be varied.

Likewise, the distal cone portion 308 is primarily configured to separate the body portion 302 from the distal collar portion 310, without limiting the scope of the disclosure. The distal cone portion 308 converges distally, along the longitudinal axis L, from the distal diametrical size D_{D} to a second diametrical size D₂. The second diametrical size D₂ is smaller than the distal diametrical size D_{D}. Convergence angle of the distal cone portion 308 may depend on several factors, such as geometrical parameters (length and diameter) of the body portion 302, material properties (e.g., elasticity, stiffness, etc.) of the expandable member 114, the diameter of the distal end section 210, etc.

The distal collar portion 310 extends distally, along the longitudinal axis L, from the second diametrical size D₂. The distal collar portion 310 is adapted to be mounted to the distal end region 216 of the inner tubular member 204. Depending on the geometrical profile of the distal end region 216 of the inner tubular member 204, the distal collar portion 310 may be configured with a substantially uniform diameter or variable diameter, along the longitudinal axis L. The distal collar portion 310 may be mounted to the distal end region 216 through a suitable leakproof technique. In one example, the distal collar portion 310 may be adhesively bonded to the distal end region 216. In another example, the distal collar portion 310 may be thermally bonded to the distal end region 216. In these examples, the specific bonding location of the distal end region 216 can be varied.

The compliance characteristics of the expandable member 114 are essential for its performance in vascular procedures of retrograde access, referring to its ability to deform and adapt in response to applied pressure. Crucial aspects include material properties, such as but not limited to elasticity, stiffness, etc., which allows the expandable member 114 to stretch and return to its original shape (i.e., inflate and deflate); distensibility, enabling the expandable member 114 to expand under pressure (radial pressure) without rupturing; and pressure response, which describes how the diameter changes with varying inflation pressures. Additionally, the compliance characteristics of the expandable member 114 are largely influenced by geometrical factors, such as wall thickness, cross-sectional dimensions, number of layers, and other related variables. In the following embodiments, these characteristics are adapted to alter the compliance characteristics for the expandable member 114 being used in the balloon catheter 102.

Based on the preceding discussion, it can be stated that the body portion 302 is a part of the expandable member 114 that carries out desired functions. These functions include, but are not limited to, dilating narrowed blood vessels, compressing plaque against the vessel walls, and delivering medications via retrograde access. Thus, the body portion 302 can be broadly considered synonymous with the expandable member 114 in the different embodiments described below.

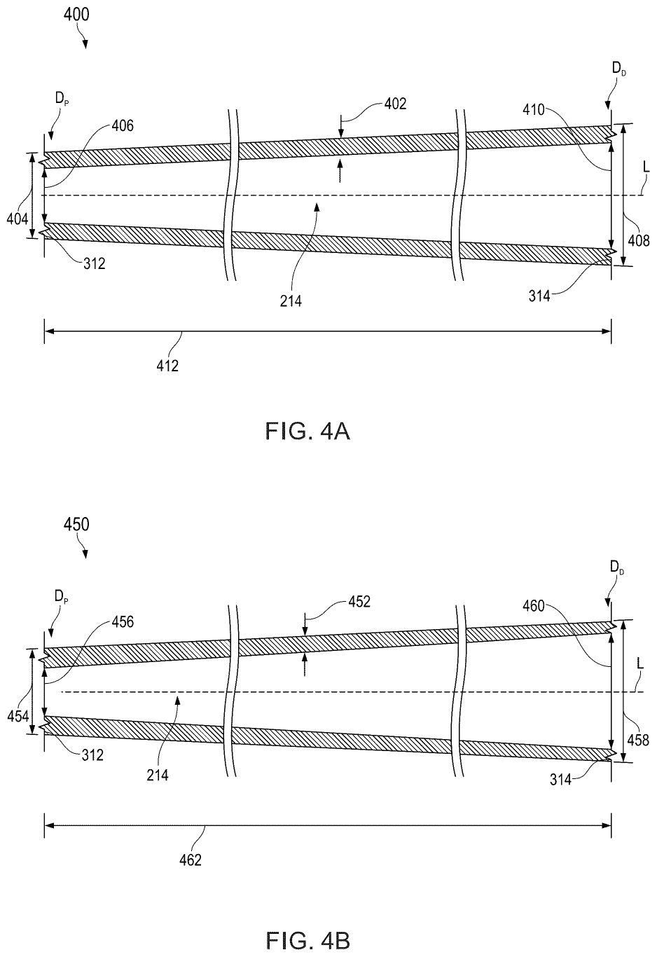

FIG. 4A illustrates a sectional view of an expandable member 400 depicted in FIGS. 3A-3B, in accordance with one embodiment of the present disclosure. The expandable member 400, as shown in FIG. 4A, is one embodiment of the expandable member 114 shown in FIGS. 1A-1B, 2A-2B, and 3A-3B. The expandable member 400, designed to conform to the natural taper (increasing diameter from a proximal region to a distal region) of the vessel (e.g., the blood vessel 106) when inserted from retrograde access, is configured with the proximal diametrical size D_{P} and the distal diametrical size D_{D}. In a specific embodiment, the distal diametrical size D_{D} is larger than the proximal diametrical size D_{P}. Therefore, the expandable member 400 increases from the proximal diametrical size D_{P} to the distal diametrical size D_{D}, along the longitudinal axis L thereof, and is adapted to dilate the body lumen 104 of the subject 100. The inflated configuration of the expandable member 400 is adapted to conform to a tapered profile of the vessel (e.g., the blood vessel 106) of the body lumen 104 when introduced through the retrograde access.

In this embodiment, a wall thickness 402 of the expandable member 400 is maintained uniformly along the longitudinal axis L. The proximal diametrical size D_{P}, at the proximal end 312, features a proximal outer diameter 404 and a proximal inner diameter 406. Likewise, the distal diametrical size D_{D}, at the distal end 314, features a distal outer diameter 408 and a distal inner diameter 410. Consequently, the difference between the proximal outer diameter 404 and the proximal inner diameter 406, and between the distal outer diameter 408 and the distal inner diameter 410 remains consistent along the longitudinal axis L. In other words, the wall thickness 402 of the expandable member 400 maintains uniform along the longitudinal axis L.

The wall thickness 402 of the expandable member 400 can be maintained uniform through various approaches. In one such approach, discussed through an example, the proximal outer diameter 404 of the proximal diametrical size D_{P} may range from 2 millimeters (mm) to 3 mm (e.g., 2.5 mm), while the distal outer diameter 408 of the distal diametrical size D_{D} may range from 3 mm to 4 mm (e.g., 3.5 mm). This implies that the body portion 302 exhibits a taper, or an increase in outer diameter, of approximately 1 mm from the proximal end 312 to the distal end 314. Similarly, the inner diameter (i.e., the proximal inner diameter 406 and the distal inner diameter 410) tapers in the same manner, ensuring that the wall thickness 402 of the expandable member 400 remains substantially uniform along the longitudinal axis L. Further, it is contemplated that a length 412 of the body portion 302 may be selected for a desired treatment location. In one example, the length may be about 300 mm. Alternatively, in some examples, the length may be less than 300 mm (e.g., 200 mm, 250 mm, etc.). Alternatively, in other examples, the length may be more than 300 mm (350 mm, 400 mm, etc.).

The uniform wall thickness 402 of the expandable member 400 is beneficial to enhance its compliance. The wall thickness 402 ensures consistent inflation characteristics, allowing for predictable inflation and controlled dilation of blood vessels (e.g., blood vessel 106), which is vital during procedures, such as angioplasty. This uniformity in thickness also supports structural integrity, reducing the likelihood of rupture or deformation under pressure. Additionally, a consistent wall thickness facilitates even stress distribution during inflation, minimizing potential weak points that could compromise performance.

FIG. 4B illustrates a sectional view of an expandable member 450 depicted in FIGS. 3A-3B, in accordance with another embodiment of the present disclosure. The expandable member 450, as shown in FIG. 4B, is one embodiment of the expandable member 114 shown in FIGS. 1A-1B, 2A-2B, and 3A-3B. The expandable member 450, designed to conform to the natural taper (increasing diameter from a proximal region to a distal region) of the vessel (e.g., the blood vessel 106) when inserted from retrograde access, is configured with the proximal diametrical size D_{P} and the distal diametrical size D_{D}. In a specific embodiment, the distal diametrical size D_{D} is larger than the proximal diametrical size D_{P}. Therefore, the expandable member 450 increases from the proximal diametrical size D_{P} to the distal diametrical size D_{D}, along the longitudinal axis L thereof, and is adapted to dilate the body lumen 104 of the subject 100. The inflated configuration of the expandable member 450 is adapted to conform to a tapered profile of the vessel (e.g., the blood vessel 106) of the body lumen 104 when introduced through retrograde access.

As the expandable member 450 is designed to conforms to the natural taper of the arteries or veins (e.g., the blood vessel 106) when inserted from the retrograde access, it is necessary for a region of the proximal diametrical size D_{P} to expand to a larger diameter than a region of the proximal diametrical size D_{D} under the same pressure. With this consideration, in this embodiment, a wall thickness 452 of the expandable member 450 reduces, along the longitudinal axis L, from the proximal diametrical size D_{P} to the distal diametrical size D_{D}. The proximal diametrical size D_{P}, at the proximal end 312, features a proximal outer diameter 454 and a proximal inner diameter 456. Likewise, the distal diametrical size D_{D}, at the distal end 314, features a distal outer diameter 458 and a distal inner diameter 460. Consequently, the difference between the proximal outer diameter 454 and the proximal inner diameter 456, and between the distal outer diameter 458 and the distal inner diameter 460 reduces distally (along the longitudinal axis L) from the proximal diametrical size D_{P} to the distal diametrical size D_{D}.

The wall thickness 452 of the expandable member 450 can be reduced through various approaches. In one such approach, a slope of an inner diameter (i.e., difference between the distal inner diameter 460 and the proximal inner diameter 456 for a length 462) increases distally, while maintaining a uniform slope of an outer diameter (i.e., difference between the distal outer diameter 458 and the proximal outer diameter 454 for the length 462). Consequently, the slope of the wall thickness 452 reduces from the proximal diametrical size D_{P} to the distal diametrical size D_{D}. The other possible approaches of reducing the wall thickness 452 from the proximal diametrical size D_{P} to the distal diametrical size D_{D} are analogous to this approach, and therefore omitted for the sake of brevity.

This configuration facilitates a more significant increase in diameter toward the distal direction when inflated and enhances the ability of the expansion member 450 to conform to the anatomy of the vessel being treated by retrograde access. In retrograde access, the reduced wall thickness 452 from the proximal diametrical size D_{P} to the distal diametrical size D_{D} enhances conformability, enabling the expansion member 450 to better adapt to the taper shape of the vessel (e.g., the blood vessel 106) and ensure optimal contact for dilation. This design results in smoother insertion and reduces trauma to surrounding tissues during procedures.

In the above embodiments, discussed with reference to FIGS. 4A and 4B, the expandable member 114/400/450 is made of a single material. In an embodiment, the expandable member 114/400/450 may be formed from compliant or semi-compliant materials, such as, but not limited to polyurethanes, silicone, polyether block amides (such as, but not limited to, PEBAX®), higher durometer polyurethanes, etc. The use of compliant and semi-compliant materials offers several benefits in retrograde access, including enhanced flexibility and controlled dilation in the region of distal diametrical size D_{D}. Compliant materials allow the expandable member 114/400/450 to adapt to the vessel's shape, improving contact and reducing trauma during inflation. Further, semi-compliant materials offer predictable expansion characteristics, ensuring precise dilation during retrograde access.

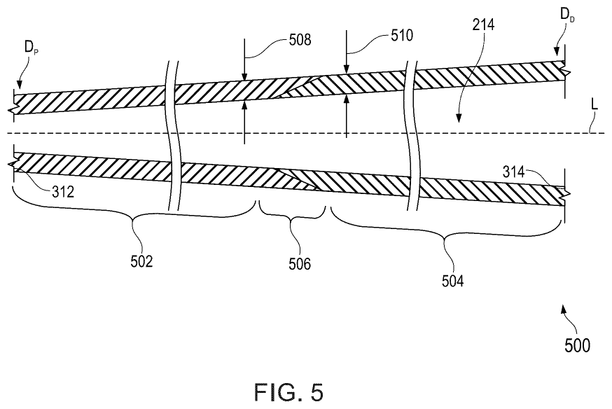

FIG. 5 illustrates a sectional view of an expandable member 500 depicted in FIGS. 3A-3B, in accordance with another embodiment of the present disclosure. The expandable member 500, as shown in FIG. 5, is one embodiment of the expandable member 114 shown in FIGS. 1A-1B, 2A-2B, and 3A-3B. The expandable member 500, designed to conform to the natural taper (increasing diameter from a proximal region to a distal region) of the vessel (e.g., the blood vessel 106) when inserted from retrograde access, is configured with the proximal diametrical size D_{P} and the distal diametrical size D_{D}. In a specific embodiment, the distal diametrical size D_{D} is larger than the proximal diametrical size D_{P}. Therefore, the expandable member 500 increases from the proximal diametrical size D_{P} to the distal diametrical size D_{D}, along the longitudinal axis L thereof, and is adapted to dilate the body lumen 104 of the subject 100. The inflated configuration of the expandable member 500 is adapted to conform to a tapered profile of the vessel (e.g., the blood vessel 106) of the body lumen 104 when introduced through retrograde access.

The expandable member 500 may include two or more different materials, along the longitudinal axis L, to vary the compliance characteristics of the inflation of the expandable member 500. In this embodiment, the material composition of the expandable member 500 may vary from the proximal end 312 (i.e., the proximal diametrical size D_{P}) to the distal end 314 (i.e., the distal diametrical size D_{D}). Specifically, the expandable member 500 includes a first length portion 502 extending distally from the proximal diametrical size D_{P}, a second length portion 504 extending proximally from the distal diametrical size D_{D}, and a transition portion 506 extending between the first length portion 502 and the second length portion 504. The transition portion 506 may include a gradient transition from the first material and the second material. The first length portion 502, the transition portion 506, and the second length portion 504 collectively define the expandable member 500.

The first length portion 502, configured as a proximal length portion of the expandable member 500, is made of a first material. On the other hand, the second length portion 504, configured as a distal length portion of the expandable member 500, is made of a second material that is different from the first material. As the expandable member 500 is designed to conforms to the natural taper of the arteries or veins (e.g., the blood vessel 106) when inserted from the retrograde access, it is necessary for the second length portion 504 to expand to a larger diameter than the proximal length portion under the same pressure. With this consideration, the second material is chosen to be softer than the first material, allowing the second length portion 504 to expand to a larger diameter than the first length portion 502 under the same operating fluid pressure. In an embodiment, the second length portion 504 may be fabricated from compliant or semi-compliant material, such as, but not limited to polyurethanes, silicone, polyether block amides (such as, but not limited to, PEBAX®), higher durometer polyurethanes, etc. In contrast, the first length portion 502 may be formed from semi-compliant or non-compliant materials such as, but not limited to, polyamides, polyesters, polyether block amides (such as, but not limited to, PEBAX® or VESTAMID®). This configuration facilitates a more significant increase in diameter toward the distal direction when inflated and enhances the ability of the expansion member 500 to conform to the anatomy of the vessel being treated by retrograde access.

Additionally, a wall thickness 508 of the first length portion 502 and/or a wall thickness 510 of the second length portion 504 may either remain consistent or vary, depending on the compliance characteristics during the inflation of the expandable member 500.

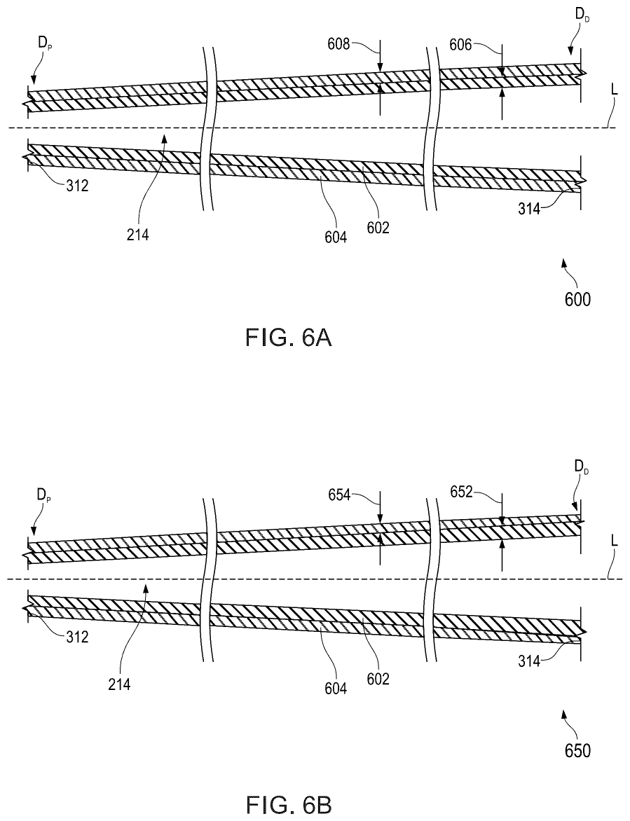

FIG. 6A illustrates a sectional view of an expandable member 600 depicted in FIGS. 3A-3B, in accordance with another embodiment of the present disclosure. The expandable member 450, as shown in FIG. 6A, is one embodiment of the expandable member 114 shown in FIGS. 1A-1B, 2A-2B, and 3A-3B. The expandable member 600, designed to conform to the natural taper (increasing diameter from a proximal region to a distal region) of the vessel (e.g., the blood vessel 106) when inserted from retrograde access, is configured with the proximal diametrical size D_{P} and the distal diametrical size D_{D}. In a specific embodiment, the distal diametrical size D_{D} is larger than the proximal diametrical size D_{P}. Therefore, the expandable member 600 increases from the proximal diametrical size D_{P} to the distal diametrical size D_{D}, along the longitudinal axis L thereof, and is adapted to dilate the body lumen 104 of the subject 100. The inflated configuration of the expandable member 600 is adapted to conform to a tapered profile of the vessel (e.g., the blood vessel 106) of the body lumen 104 when introduced through retrograde access.

The expandable member 600 of the present embodiment is made of two or more layers, including but not limited to, an inner layer 602 and an outer layer 604 disposed on the inner layer 602. While a wall thickness 606 of the inner layer 602 and a wall thickness 608 of the outer layer 604 remain uniform along the longitudinal axis L. In a non-limiting example, the inner layer 602 and the outer layer 604 may be co-extruded as a multi-layer co-extrusion. It is contemplated that the inner layer 602 and the outer layer 604 may be formed from different materials to vary compliance characteristics of the expandable member 600 along the longitudinal axis L thereof. Additionally, or alternatively, compliance characteristics of the expandable member 600 may be varied by changing wall thickness, cross-sectional dimensions, and/or material type of one or more layers 602, 604 along the longitudinal axis L.

As the expandable member 600 is designed to conforms to the natural taper of the arteries or veins (e.g., the blood vessel 106) when inserted from the retrograde access, it is necessary for a region of the proximal diametrical size D_{P} to expand to a larger diameter than a region of the proximal diametrical size D_{D} under the same pressure. With this consideration, in the present embodiment, the outer layer 604 may be formed from a first material and the inner layer 602 may be formed from a second material, wherein the second material is different from the first material. It is contemplated that the second material may be softer than the first material. In other words, the inner layer 602 is more compliant than the outer layer 604. The inner layer 602 may be formed from compliant or semi-compliant materials, such as, but not limited to polyurethanes, silicone, polyether block amides (such as, but not limited to, PEBAX®), higher durometer polyurethanes, etc. Correspondingly, the outer layer 604 may be formed from semi-compliant or non-compliant materials such as, but not limited to, polyamides, polyesters, and polyether block amides (such as, but not limited to, PEBAX® or VESTAMID®). In some examples, either or both of the inner or outer layers 602, and 604 may transition from a first material to a second material along the longitudinal axis L, like that described with respect to FIG. 5. In other words, the material composition of the inner layer 602 and/or the outer layer 604 may vary along the longitudinal axis L. This configuration facilitates a more significant increase in diameter toward the distal direction when inflated and enhances the ability of the expansion member 600 to conform to the anatomy of the vessel being treated by retrograde access.

FIG. 6B illustrates a sectional view of an expandable member 650 depicted in FIGS. 3A-3B, in accordance with another embodiment of the present disclosure. The expandable member 600, as shown in FIG. 6B, is one embodiment of the expandable member 114 shown in FIGS. 1A-1B, 2A-2B, and 3A-3B. The expandable member 650, designed to conform to the natural taper (increasing diameter from a proximal region to a distal region) of the vessel (e.g., the blood vessel 106) when inserted from retrograde access, is configured with the proximal diametrical size D_{P} and the distal diametrical size D_{D}. In a specific embodiment, the distal diametrical size D_{D} is larger than the proximal diametrical size D_{P}. Therefore, the expandable member 650 increases from the proximal diametrical size D_{P} to the distal diametrical size D_{D}, along the longitudinal axis L thereof, and is adapted to dilate the body lumen 104 of the subject 100. The inflated configuration of the expandable member 650 is adapted to conform to a tapered profile of the vessel (e.g., the blood vessel 106) of the body lumen 104 when introduced through retrograde access.

The expandable member 650 of the present embodiment is made of two or more layers, including but not limited to, the inner layer 602 and the outer layer 604 disposed on the inner layer 602. Additionally, ratio of a wall thickness 652 of the inner layer 602 to a wall thickness 654 of the outer layer 604 increases, along the longitudinal axis L, from the proximal diametrical size D_{P} to the distal diametrical size D_{D} to control the expansion profile of the expansion member 600. This configuration facilitates a more significant increase in diameter toward the distal direction when inflated and enhances the ability of the expansion member 650 to conform to the anatomy of the vessel being treated by retrograde access.

As the expandable member 650 is designed to conforms to the natural taper of the arteries or veins (e.g., the blood vessel 106) when inserted from the retrograde access, it is necessary for a region of the proximal diametrical size D_{P} to expand to a larger diameter than a region of the proximal diametrical size D_{D} under the same pressure. With this consideration, in the representative example, a distal region of the expandable member 650 having a higher ratio of the wall thickness 652 of the inner layer 602 to the wall thickness 654 of the outer layer 604 may expand to a greater extent than a proximal region of the expandable member 650 having a lower ratio of the wall thickness 652 of the inner layer 602 to the wall thickness 654 of the outer layer 604. In other words, the outer layer 604 (i.e., lesser compliant material), having higher thickness, limits the radial expansion of the expandable member 650 to a greater extent than the outer layer 604 having lesser thickness. Therefore, to distally increase the radial growth of the expandable member 600, the wall thickness 652 of the inner layer 602 may increase distally, while the wall thickness 654 of the outer layer 604 may increase proximally. The other possible approaches of increasing the ratio of the wall thickness 652 of the inner layer 602 to the wall thickness 654 of the outer layer 604, along the longitudinal axis L, are analogous to this approach, and therefore omitted for the sake of brevity.

In each of the embodiments, the inflated configuration of the expandable member 114/400/450/500/600/650 defines the shape of a diverging frustum extending from the proximal diametrical size D_{P} to the distal diametrical size D_{D}, along the longitudinal axis L. The diverging frustum shape is beneficial, as it allows for optimal contact with the vessel walls, through retrograde access, during dilation, providing effective expansion and minimizing the risk of vessel trauma. Additionally, this geometrical feature facilitates better flow dynamics while improving the overall performance and effectiveness of the catheter 102 during procedures.

Without limiting the scope of the disclosure, the expandable member 114/400/450/500/600/650 is manufactured using a blow molding process. The molding process involves inflating a heated thermoplastic material within a mold to form the required shape. This technique allows for precise control over the dimensions and wall thickness of the expandable member 114/400/450/500/600/650, resulting in a uniform structure thereof.

Further, it is contemplated that the expandable member 114/400/450/500/600/650 may be affixed with one or more cutting blades or scoring members (not shown). More specifically, the cutting blades or scoring members can be strategically spaced along the length and circumference of the expandable member 114/400/450/500/600/650 allowing for targeted vascular interventions that enable the balloon to cut through or score the tissue of narrowed or obstructed vessels. Additionally, or alternatively, the expansion member 114/400/450/500/600/650 may be coated with a therapeutic agent, such as but not limited to anti-thrombogenic agents including heparin, heparin derivatives, urokinase, etc.

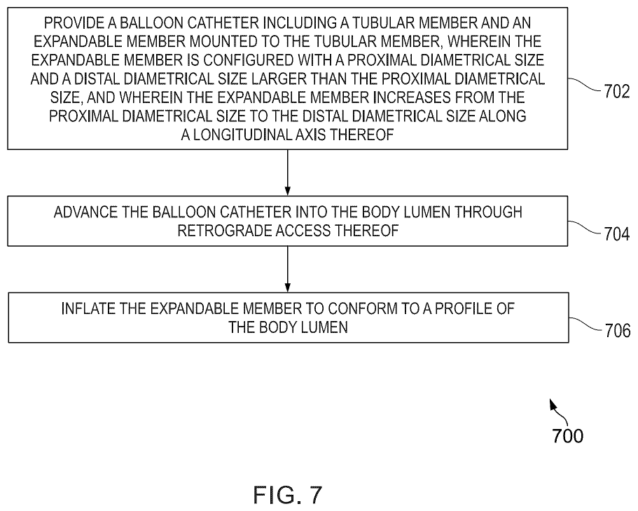

FIG. 7 illustrates a flow diagram of a method 700 for dilating the body lumen 104, in accordance with various embodiments of the present disclosure. It is to be noted that the sequence of the method 700 may not be necessarily executed in the same order as they are presented. Further, one or more steps may be grouped and performed in the form of a single step, or one step may have several sub-steps that may be performed in a parallel or a sequential manner. The method 700 begins at step 702.

At step 702, the method 700 includes providing the balloon catheter 102. The balloon catheter 102 mainly includes the catheter body 112, the expandable member 114/400/450/500/600/650 adapted to be mounted to the catheter body 112, and the guidewire 116 adapted to extend at least partially through the catheter body 112 and fully through the expandable member 114. The geometrical configuration and design features of each of the catheter body 112, the expandable member 114/400/450/500/600/650, and the guidewire 116 have already been discussed above with reference to FIGS. 1A-1B to 3A-3B, and therefore not reiterated here for the sake of brevity.

Next, retrograde access to the body lumen 104 via the vascular access point 108 is obtained. To obtain retrograde access to the body lumen 104 via the vascular access point 108, the process begins with preparing the patient 100 and sterilizing the access site. Upon obtaining the retrograde access, the balloon catheter 102 is inserted into the vascular access point 108.

Further, step 704 includes advancing the balloon catheter 102 into the body lumen 104 through retrograde access thereof. Further, fluid is introduced into the chamber 214 of the expandable member 114/400/450/500/600/650. The chamber 214 fluidically communicates with the inflation lumen 206 and enables the expandable member 114/400/450/500/600/650 to transition between the inflated configuration and the deflated configuration thereof. The inflation lumen 206 can supply an inflation medium (i.e., fluid) within the chamber 214 under positive pressure. In contrast, the inflation lumen 206 may receive the inflation medium from the chamber 214 under negative pressure (i.e., vacuum pressure). The expandable member 114/400/450/500/600/650 can thus be inflated and deflated, depending on the fluid supplied or extracted from the chamber 214.

At step 706, inflating the expandable member to conform to a profile of the body lumen. Finally the expandable member 114/400/450/500/600/650 is deflated and the balloon catheter 102 from the body lumen 104.

Various embodiments of the disclosure, as discussed above, may be practiced with steps and/or operations in a different order, and/or with hardware elements in configurations, which are different than those which, are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A balloon catheter, comprising:
a tubular member defining an inflation lumen therein at least to facilitate a fluid flow, the tubular member comprising:
a proximal end section adapted to be fluidically coupled to a fluid source; and
a distal end section adapted to be introduced into a body lumen at least through retrograde access thereof; and
an expandable member mounted to the distal end section and defining a chamber therein, wherein the chamber fluidically communicates with the inflation lumen and enables the expandable member to transition between an inflated configuration and a deflated configuration,
wherein the expandable member is configured with a proximal diametrical size and a distal diametrical size larger than the proximal diametrical size, the expandable member increasing from the proximal diametrical size to the distal diametrical size along a longitudinal axis thereof and adapted to dilate the body lumen in the inflated configuration.

2. The balloon catheter as claimed in claim 1, wherein the expandable member comprises:
a body portion comprising a proximal end and a distal end, wherein the proximal end is configured with the proximal diametrical size, and wherein the distal end is configured with the distal diametrical size larger than the proximal diametrical size;
a proximal conical portion converging proximally, along the longitudinal axis, from the proximal diametrical size to a first diametrical size thereof, wherein the first diametrical size is smaller than the proximal diametrical size;
a proximal collar portion extending proximally, along the longitudinal axis, from the first diametrical size, wherein the proximal collar portion is adapted to be mounted to the distal end section;
a distal conical portion converging distally, along the longitudinal axis, from the distal diametrical size to a second diametrical size thereof, wherein the second diametrical size is smaller than the distal diametrical size; and
a distal collar portion extending distally, from the second diametrical size, along the longitudinal axis.

3. The balloon catheter as claimed in claim 2, wherein the tubular member is configured as an outer tubular member, and wherein the balloon catheter further comprises:
an inner tubular member extending at least partially through the tubular member and fully through the expandable member, wherein the inner tubular member defines a guidewire lumen therein, and wherein the inner tubular member and the tubular member defines the inflation lumen there between, the inner tubular member comprising:
a proximal end region; and
a distal end region adapted at least to mount the distal collar portion; and
a guidewire adapted to be inserted from the proximal end region, passing through the guidewire lumen, and extracted from the distal end region.

4. The balloon catheter as claimed in claim 1, wherein a wall thickness of the expandable member is uniform along the longitudinal axis.

5. The balloon catheter as claimed in claim 1, wherein a wall thickness of the expandable member reduces along the longitudinal axis from the proximal diametrical size to the distal diametrical size.

6. The balloon catheter as claimed in claim 1, wherein the expandable member is made of a single material.

7. The balloon catheter as claimed in claim 1, wherein the expandable member comprises:
a first length portion extending distally from the proximal diametrical size, wherein the first length portion is made of a first material;
a second length portion extending proximally from the distal diametrical size, wherein the second length portion is made of a second material different from the first material; and
a transition portion extending between the first length portion and the second length portion,
wherein the first length portion, the transition portion, and the second length portion collectively define the expandable member.

8. The balloon catheter as claimed in claim 7, wherein the second material is more compliant than the first material.

9. The balloon catheter as claimed in claim 7, wherein the transition portion comprises a gradient transition from the first material and the second material.

10. The balloon catheter as claimed in claim 1, wherein the expandable member comprises an inner layer and an outer layer disposed on the inner layer.

11. The balloon catheter as claimed in claim 10, wherein the outer layer is made of a first material and the inner layer is made of a second material, wherein the second material is more compliant than the first material.

12. The balloon catheter as claimed in claim 10, wherein a wall thickness of the outer layer and a wall thickness of the inner layer are substantially uniform along the longitudinal axis.

13. The balloon catheter as claimed in claim 10, wherein a ratio of a wall thickness of the inner layer to a wall thickness of the outer layer increases, along the longitudinal axis, from the proximal diametrical size to the distal diametrical size.

14. The balloon catheter as claimed in claim 1, wherein the inflated configuration of the expandable member is adapted to conform to a tapered profile of a vessel of the body lumen when introduced through the retrograde access.

15. The balloon catheter as claimed in claim 1, wherein the inflated configuration of the expandable member defines the shape of a diverging frustum extending from the proximal diametrical size to the distal diametrical size.

16. The balloon catheter as claimed in claim 1, wherein the expandable member is manufactured using a blow molding process.

17. A method for dilating a body lumen, comprising:
providing a balloon catheter comprising a tubular member and an expandable member mounted to the tubular member, wherein the expandable member is configured with a proximal diametrical size and a distal diametrical size larger than the proximal diametrical size, and wherein the expandable member increases from the proximal diametrical size to the distal diametrical size along a longitudinal axis thereof;
advancing the balloon catheter into the body lumen through retrograde access thereof; and
inflating the expandable member to conform to a profile of the body lumen.

18. The method as claimed in claim 17, further comprising:
introducing fluid into a chamber of the expandable member;
deflating the expandable member; and
withdrawing the balloon catheter from the body lumen.

19. The method as claimed in claim 17, further comprising:
obtaining retrograde access to the body lumen via a vascular access point; and
inserting the balloon catheter into the vascular access point.

20. The method as claimed in claim 17, wherein the expandable member comprises:
a body portion comprising a proximal end and a distal end, wherein the proximal end is configured with the proximal diametrical size, and wherein the distal end is configured with the distal diametrical size larger than the proximal diametrical size ;
a proximal conical portion converging proximally, along the longitudinal axis, from the proximal diametrical size to a first diametrical size thereof, wherein the first diametrical size is smaller than the proximal diametrical size;
a proximal collar portion extending proximally, along the longitudinal axis, from the first diametrical size, wherein the proximal collar portion is adapted to be mounted to the distal end section ;
a distal conical portion converging distally, along the longitudinal axis, from the distal diametrical size to a second diametrical size thereof, wherein the second diametrical size is smaller than the distal diametrical size; and
a distal collar portion extending distally, from the second diametrical size, along the longitudinal axis.
